(19) United States
(12) Patent Application Publication
(10) Pub. No.: US 2021/0173191 A1
(43) Pub. Date: Jun. 10, 2021

(54) SYSTEMS AND METHODS FOR ACTIVE VIBRATION REDUCTION OF A SURGICAL MICROSCOPE

(71) Applicant: Alcon Inc., Fribourg (CH)

(72) Inventor: Jiansheng Zhou, Cerritos, CA (US)

(21) Appl. No.: 17/181,341

(22) Filed: Feb. 22, 2021

Related U.S. Application Data
(63) Continuation of application No. 15/817,874, filed on Nov. 20, 2017.
(60) Provisional application No. 62/436,273, filed on Dec. 19, 2016.

(51) Int. Cl. G02B 21/24; G02B 27/64; G01P 15/18; G02B 21/00; A61B 90/20; G01P 13/00; A61B 3/13; G02B 7/00; A61B 34/20

(52) U.S. Cl. CPC G02B 21/241; G02B 27/646; G01P 15/18; G02B 21/0012; A61B 2034/2048; G01P 13/00; A61B 3/13; G02B 7/001; A61B 90/20

(57) ABSTRACT

The present disclosure provides a system for active vibration reduction of a surgical microscope. The system includes a surgical microscope with an optical head connected to a control device that adjusts the position of the optical head in any of the X, Y, and Z-directions. The system also includes a processor and an accelerometer that detects motion of the optical head and generates data relating to the motion. The disclosure also provides a method for active vibration reduction, which includes receiving data relating to motion of an optical head of a surgical microscope, determining whether the motion is a vibration event or an intentional movement, determining a distance and direction the optical head must be adjusted to reduce the vibration caused by the motion detected, and generating and transmitting a control signal to adjust the position of the optical head to reduce the vibration of the vibration event.

### TECHNICAL FIELD

The disclosure relates to ophthalmic surgery, and more specifically, to active vibration reduction of a surgical microscope.

### BACKGROUND

Eye surgery, or ophthalmic surgery, saves and improves the vision of tens of thousands of patients every year. However, given the sensitivity of vision to even small changes in the eye and the minute and delicate nature of many eye structures, ophthalmic surgery is difficult to perform and the reduction of even minor or uncommon surgical errors or modest improvements in accuracy of surgical techniques can make an enormous difference in the patient's vision after the surgery.

Ophthalmic surgery is performed on the eye and accessory visual structures. During ophthalmic surgery, a patient is placed on a support, facing upward, under a surgical microscope. An eye speculum is inserted to keep the eye exposed. Surgeons often use a surgical microscope to view the patient's eye, and surgical instruments may be introduced to perform any of a variety of different procedures. The surgical microscope provides imaging and optionally illumination of parts of the eye during the procedure. A surgical microscope may be configured in many forms, for example, a ceiling-mounted surgical microscope or a mobile cart-mounted surgical microscope.

When operating a surgical microscope, it is important for the optical head of the surgical microscope to stay relatively still so that the user can accurately observe the eye. During normal operation, the optical head may vibrate and hinder the focus of the surgical microscope. This may occur, for example, when the user adjusts the position of the optical head or accidently contacts a component of the surgical microscope. The optical head may also vibrate due to ambient conditions in the room, such as air flow, or ambient conditions affecting the room, such as vibration caused by other medical equipment nearby, vibration of the ground or the structure of the building in which the surgical microscope is operated. Significant vibration may render a surgical microscope inoperable for accurately observing the eye.

### SUMMARY

The present disclosure provides a system for active vibration reduction. The system includes a surgical microscope with an optical head connected to a control device, the control device operable to adjust a position of the optical head, an accelerometer connected to the optical head, the accelerometer operable to detect motion of the optical head and generate data relating to the motion, and a processor configured to determine whether the motion is a vibration event or an intentional movement, determine the distance and direction the optical head must be adjusted to reduce vibration of the vibration event, when a vibration event is determined to occur, generate a control signal, the control signal operable to adjust a position of the optical head in the distance and direction determined, and transmit the control signal to the control device.

In additional embodiments, which may be combined with one another unless clearly exclusive: the accelerometer is a 3-axis accelerometer that detects and generates data relating to the motion of the optical head in the X, Y, and Z-directions; the accelerometer is an accelerometer that detects and generates data relating to the motion of the optical head in at least one of the X, Y, and Z-directions; determining whether the motion is a vibration event or an intentional movement includes determining whether a displacement caused by the motion, after a first motion reversal, is greater than an active reduction cutoff and less than a normal vibration boundary, and wherein the vibration event is a momentary external vibration event when the displacement is greater than the active reduction cutoff and less than the normal vibration boundary; a control signal is only generated when the motion is determined to be the momentary external vibration event; the control signal is further operable to adjust the position of the optical head in any of the X, Y, and Z-directions; the position of the optical head in any of the X, Y, and Z-directions is adjusted in the direction opposite the direction of the motion detected in each of the X, Y, and Z-directions; the position of the optical head is adjusted only until the displacement of the optical head is reduced below the active reduction cutoff; determining whether the motion is a vibration event or an intentional movement includes determining whether a displacement caused by the motion is less than an ambient vibration boundary, and wherein the vibration event is an ambient vibration event when the displacement is less than the ambient vibration boundary; a control signal is only generated when the motion is determined to be the ambient vibration event; the control signal is further operable to adjust the position of the optical head in any of the X, Y, and Z-directions; the position of the optical head in any of the X, Y, and Z-directions is adjusted in the direction opposite the direction of the motion detected in each of the X, Y, and Z-directions; the position of the optical head is adjusted only until displacement of the optical head is reduced below a user-selected reduction threshold; and determining the distance and direction the optical head must be adjusted to reduce the vibration detected, generating, and transmitting the control signal is performed in real time.

The disclosure also provides a method for performing active vibration reduction of a surgical microscope. The method includes receiving data relating to motion of an optical head of a surgical microscope, determining whether the motion is a vibration event or an intentional movement, determining a distance and direction the optical head must be adjusted to reduce vibration of the vibration event, when a vibration event is determined to occur, generating a control signal, the control signal operable to adjust a position of the optical head in the distance and direction determined, and transmitting the control signal to a control device.

In additional embodiments, which may be combined with one another unless clearly exclusive: receiving data relating to motion of an optical head of a surgical microscope includes receiving data relating to motion in an X, Y, and Z-direction; determining whether the motion is a vibration event or an intentional movement includes determining whether a displacement caused by the motion, after a first motion reversal, is greater than an active reduction cutoff and less than a normal vibration boundary, and wherein the vibration event is a momentary external vibration event when the displacement is greater than the active reduction cutoff and less than the normal vibration boundary; a control signal is only generated when the motion is determined to be the momentary external vibration event, the control signal operable to adjust the position of the optical head in any of the X, Y, and Z-directions; the position of the optical head in any of the X, Y, and Z-directions is adjusted in the direction opposite the direction of the motion detected in each of the X, Y, and Z-directions; the position of the optical head is adjusted only until the displacement of the optical head is reduced below the active reduction cutoff; determining whether the motion is a vibration event or an intentional movement includes determining whether a displacement caused by the motion is less than an ambient vibration boundary, and wherein the vibration event is an ambient vibration event when the displacement is less than the ambient vibration boundary; a control signal is only generated when the motion is determined to be the ambient vibration event, the control signal operable to adjust the position of the optical head in any of the X, Y, and Z-directions; the position of the optical head in any of the X, Y, and Z-directions is adjusted in the direction opposite the direction of the motion detected in each of the X, Y, and Z-directions; the position of the optical head is adjusted only until displacement of the optical head is reduced below a user-selected reduction threshold; and determining the distance and direction the optical head must be adjusted to reduce the vibration detected, generating, and transmitting the control signal is performed in real time.

The above systems may be used with the above methods and vice versa. In addition, any system described herein may be used with any method described herein and vice versa.

### BRIEF DESCRIPTION OF THE DRAWINGS

For a more complete understanding of the present invention and its features and advantages, reference is now made to the following description, taken in conjunction with the accompanying drawings, which are not drawn to scale, and in which:

FIG. 1 is a schematic representation of a system for active vibration reduction of a surgical microscope;

FIG. 2 is a graph of optical head displacement versus time for motion caused by an ambient vibration event;

FIG. 3 is a graph of optical head displacement versus time for motion caused by a momentary external vibration event; and

FIG. 4 is a flowchart of a method for active vibration reduction.

### DETAILED DESCRIPTION

In the following description, details are set forth by way of example to facilitate discussion of the disclosed subject matter. It should be apparent to a person of ordinary skill in the field, however, that the disclosed embodiments are exemplary and not exhaustive of all possible embodiments.

This disclosure provides systems and methods for active vibration reduction of a surgical microscope to reduce vibration that disturbs the focus of the surgical microscope, or in some cases, may render it inoperable. The systems include an accelerometer connected to the optical head of the surgical microscope and a control device connected to the optical head. The accelerometer detects motion of the optical head in any of the X, Y, and Z-directions and generates data relating to the motion. The X and Y-directions may be defined in the plane roughly perpendicular to the apex of the surgical microscope's curved lens. The Z-direction may be defined in the plane roughly perpendicular to the plane in which the X and Y-directions are defined. The accelerometer may be, for example, a 3-axis accelerometer that detects and generates data relating to the motion of the optical head in each of the X, Y, and Z-directions. The accelerometer may also be a 1-axis or 2-axis accelerometer that detects and generates data relating to the motion of the optical head in at least one of the X, Y, and Z-directions.

A processor determines whether the motion detected is a vibration event or an intentional movement, using the data. If the motion is determined to be an intentional movement, then the processor does not send a control signal or otherwise does not cause the position of the optical head to be changed. In contrast, if the motion is determined to be a vibration event, the processor may also determine the distance and direction the optical head must be adjusted to reduce the vibration detected and send an appropriate control signal. Generally, the vibration detected may be reduced by adjusting the position of the optical head, via the control device, in a direction opposite the direction of the vibration detected, in any of the X, Y, and Z-directions. The processor generates a control signal to adjust the position of the optical head in the distance and direction determined, and transmits the control signal to the control device. By performing active vibration reduction, the systems and methods optimize the focus of the surgical microscope.

Generally, there are two types of vibration of concern when operating a surgical microscope: (1) ambient vibration; and (2) momentary external vibration.

Ambient vibration produces a small displacement vibration that may stay relatively constant or may gradually decrease with time. Ambient vibration may occur with or without a known cause. Often, it may be caused by ambient conditions in the room, such as air flow, or ambient conditions affecting the room, such as vibration caused by other medical equipment nearby, vibration of the ground or the structure of the building in which the surgical microscope is operated.

Momentary external vibration is generally caused by an external input that shakes the surgical microscope, producing a large displacement vibration that gradually decreases with time. For example, momentary external vibration may be caused by accidental contact with a component of the surgical microscope, such as the cart base of a mobile cart-mounted surgical microscope or the ceiling arm of a ceiling-mounted surgical microscope.

By performing active vibration reduction, the systems and methods disclosed herein may reduce vibration in any of the X, Y, and Z-directions and may operate at low frequencies, for example, below 10 Hz. In contrast, passive vibration reduction often involves use of vibration isolators, such as rubber or foam supports that can only reduce vibration in one direction and are often ineffective at low frequency. Because vibration isolators are typically a soft material designed to absorb an impact or vibration, they may allow the optical head to lean or tilt during unbalanced weight distribution, and swing unrestrained during intentional movement, neither of which is desirable. Finally, vibration isolator materials are selected to work with a specific weight and may require replacement or reinforcement when the weight changes, for example, if additional components are added to the optical head or the arm supporting the optical head.

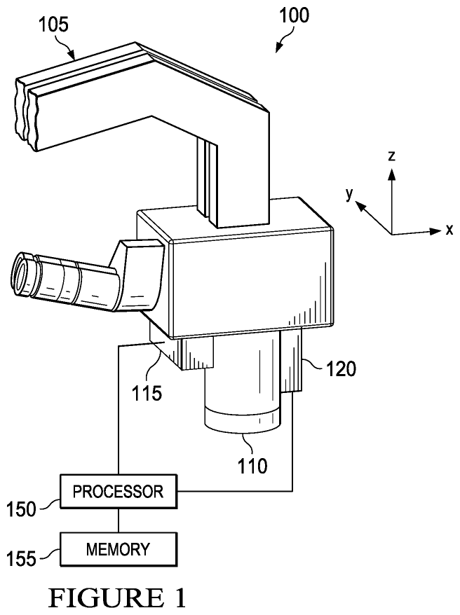

Referring now to the figures, FIG. 1 is a system 100 for active vibration reduction of the optical head 110 of a surgical microscope 105. System 100 also includes accelerometer 115 and control device 120, both of which are connected to optical head 110 and processor 150. Processor 150 is connected to memory 155.

Accelerometer 115 detects motion of optical head 110 in any of the X, Y, and Z-directions and generates data relating to the motion. Processor 150 determines whether the motion detected is a vibration event or an intentional movement, using the data and a process. If the motion is determined to be an intentional movement, processor 150 may be configured to not send a control signal or otherwise not cause the position of the optical head to be changed. In contrast, if the motion is determined to be a vibration event and not an intentional movement, processor 150 may determine the distance and direction the optical head must be adjusted to reduce the vibration, of the vibration event detected.

As described above, a vibration event may be an ambient vibration event or a momentary external vibration event. An intentional movement may be, for example, release of a brake or a lock on the surgical microscope, user-initiated movement of the surgical microscope's stage, or user-initiated movement of the optical head.

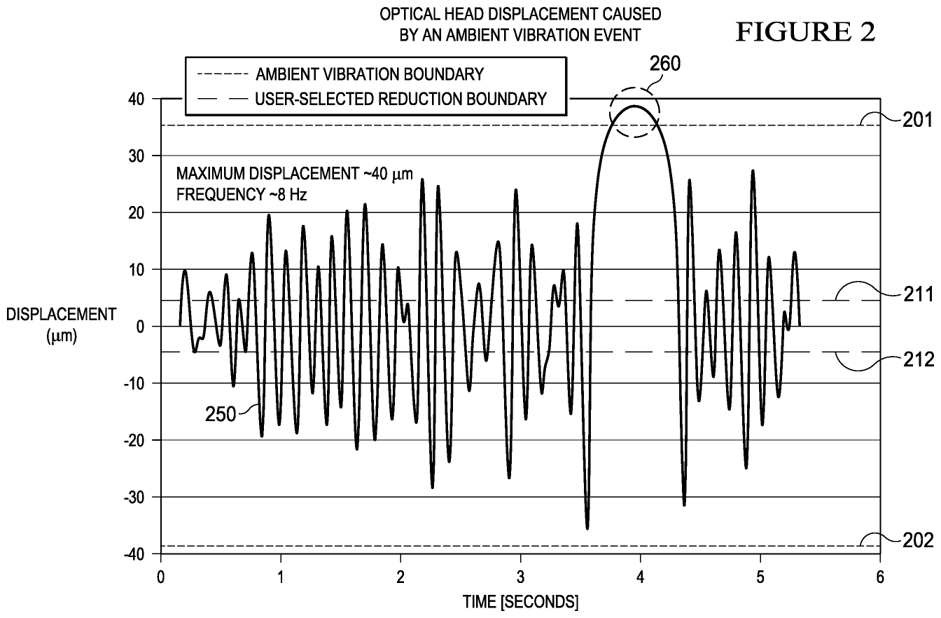

To determine whether the motion detected is an ambient vibration event, processor 150 determines whether the displacement of optical head 110, caused by the motion, is greater than an ambient vibration boundary, as illustrated in FIG. 2. If the displacement exceeds an ambient vibration boundary, it is an intentional movement. If the displacement is less than an ambient vibration boundary, it is a vibration event.

FIG. 2 is a graph of optical head displacement versus time for motion caused by an ambient vibration event. Line 250 indicates displacement, across time, of the optical head due to the motion detected by the accelerometer. Optical head displacement (in μm) is shown on the Y-axis and time (in seconds) is shown on the X-axis. In this example, ambient vibration boundaries 201 and 202 are shown at about +40 μm and −40 μm displacement. At area 260, the displacement is greater than ambient vibration boundary 201, and is therefore considered an intentional movement. However, the rest of line 250 is within ambient vibration boundaries 201 and 202, and is therefore considered an ambient vibration event.

Once processor 150 determines an ambient vibration event has occurred, processor 150 may determine the distance and direction optical head 110 must be adjusted to reduce the vibration detected. The ambient vibration may be reduced or cancelled out by adjusting the position of the optical head in a direction opposite the direction of the motion detected, in any of the X, Y, and Z-directions. Processor 150 may generate a control signal to adjust the position of the optical head in the distance and direction determined, and transmit the control signal to control device 120. The control signal generated may cause control device 120 to adjust the position of the optical head in the opposite direction of the motion detected, until the displacement caused by the motion detected is reduced below a user-selected reduction threshold, for example, user-selected reduction thresholds 211 or 212. Control device 120 may be, for example, an active motion device for X, Y, and Z-directions which can be driven by DC brush motor, DC brushless servo motor, Stepper motor, electrical solenoid, piezo actuator, or a similar device.

Processor 150 may determine the distance and direction the optical head must be adjusted to reduce the vibration detected, generate, and transmit the control signal in real time. Real time may mean in less than half a second, in less than one second, or otherwise in less than the normal reaction time of a user of the visual information.

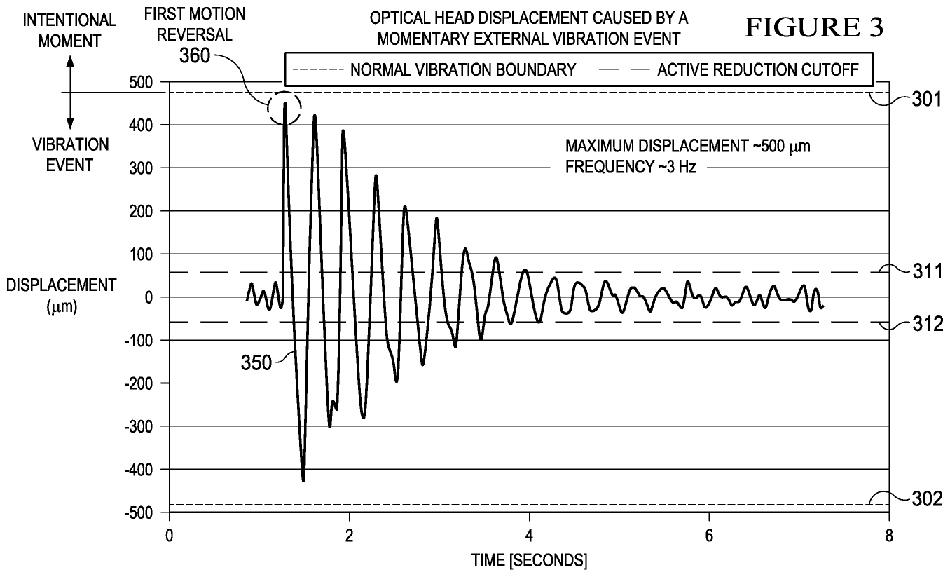

To determine whether the motion detected is a momentary external vibration event, processor 150 determines whether the displacement of optical head 110, caused by the motion, is greater than an active reduction cutoff and also less than a normal vibration boundary, as illustrated in FIG. 3. To make this determination, processor 150 compares the displacement to the active reduction cutoff and normal vibration boundary, but only after the first motion reversal. The first motion reversal, is the first time a detected motion changes direction within a single detected-motion event.

FIG. 3 is a graph of optical head displacement versus time for motion caused by a momentary external vibration event. Line 350 indicates displacement, across time, of the optical head due to the motion detected by the accelerometer. Optical head displacement (in μm) is shown on the Y-axis and time (in seconds) is shown on the X-axis. In this example, normal vibration boundaries 301 and 302 are shown at about +500 μm and −500 μm displacement. Area 360 indicates the first motion reversal of line 350. As shown, the displacement after the first motion reversal is less than about +500 μm and greater than about −500 μm, so this motion between the normal vibration boundaries is considered a momentary external vibration event. In contrast, if a displacement, after the first motion reversal, exceeds a normal vibration boundary the motion is an intentional movement.

Once processor 150 determines a momentary external vibration event has occurred, processor 150 may determine the distance and direction optical head 110 must be adjusted to reduce the vibration detected. The momentary external vibration may be reduced or cancelled out by adjusting the position of the optical head in a direction opposite the direction of the motion detected, in any of the X, Y, and Z-directions. Processor 150 may generate a control signal to adjust the position of the optical head in the distance and direction determined, and transmit the control signal to control device 120. The control signal generated may cause control device 120 to adjust the position of the optical head in the opposite direction of the motion detected, until the displacement caused by the motion detected is reduced below a user-selected active reduction cutoff, for example, user-selected active reduction cutoff 311 or 312.

Processor 150 may be configured to adjust the position of the optical head only until the displacement of the optical head is reduced below the active reduction cutoff, or any other threshold the user selects. Processor 150 may determine the distance and direction the optical head must be adjusted to reduce the vibration detected, generate, and transmit the control signal in real time.

A processor 150 may include, for example a microprocessor, microcontroller, digital signal processor (DSP), application specific integrated circuit (ASIC), or any other digital or analog circuitry configured to interpret and/or execute program instructions and/or process data. In some embodiments, processor 150 may interpret and/or execute program instructions and/or process data stored in memory 155. Memory 155 may be configured in part or whole as application memory, system memory, or both. Memory 155 may include any system, device, or apparatus configured to hold and/or house one or more memory modules. Each memory module may include any system, device or apparatus configured to retain program instructions and/or data for a period of time (e.g., computer-readable media). The various servers, electronic devices, or other machines described may contain one or more similar such processors or memories for storing and executing program instructions for carrying out the functionality of the associated machine.

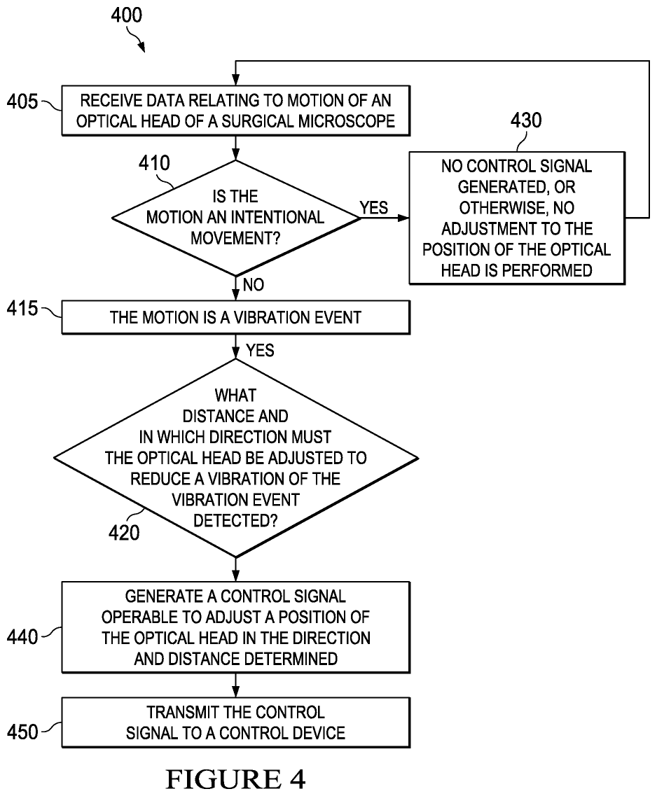

FIG. 4 is a flowchart of a method 400 for active vibration reduction. At step 405, data relating to motion of the optical head of a surgical microscope is received. The data received may include data relating to the motion in any of the X, Y, and Z-directions. At step 410, whether the motion is an intentional movement is determined. If the motion is an intentional movement, at step 430, no control signal is generated and the position of the optical head is otherwise not changed. When motion is determined to be an intentional movement, after step 430, data relating to motion of an optical head of a surgical microscope is received at step 405. In contrast, if the motion is not an intentional movement, then as shown at step 415, it is considered a vibration event, and at step 420, the distance and direction the optical head must be adjusted to reduce the vibration, of the vibration event, using the data received.

To determine whether the motion detected is a momentary external vibration event, the displacement of the optical head after the first motion reversal, caused by the motion, may be compared to an active reduction cutoff and a normal vibration boundary. If the displacement after the first motion reversal is greater than the active reduction cutoff and also less than the normal vibration boundary, the motion is a momentary external vibration event. The first motion reversal, is the first time a detected motion changes direction within a single detected-motion event. In contrast, if the displacement after the first motion reversal exceeds a normal vibration boundary, the motion is an intentional movement, and at step 430, no control signal is sent or the position of the optical head is otherwise not changed. As illustrated in FIG. 3, normal vibration boundaries may be selected by the user, for example, about +500 μm and −500 μm. In this example, if the displacement of the optical head, after the first motion reversal, is less than about +500 μm or greater than about −500 μm, the motion is a momentary external vibration event.

To determine whether the motion detected is an ambient vibration event, the displacement of the optical head, caused by the motion, may be compared to an ambient vibration boundary. If the displacement is less than an ambient vibration boundary, the motion is an ambient vibration event. As illustrated in FIG. 2, ambient vibration boundaries may be selected by the user, for example, about +40 μm and −40 μm. In this example, if the displacement of the optical head is between about +40 μm and −40 μm, the motion is an ambient vibration event. In contrast, if the displacement of the optical head is greater than about +40 μm or less than about −40 μm, exceeding one of the ambient vibration boundaries, the motion is intentional, and at step 430, no control signal is sent or the position of the optical head is otherwise not changed.

At step 440, a control signal may be generated to adjust a position of the optical head in the direction and distance determined at step 420. At step 450, the control signal may be transmitted to a control device to adjust the position of the optical head.

The control signal may adjust the position of the optical head in any of the X, Y, and Z-directions. For example, the control signal may adjust the position of the optical head in the direction opposite the direction of the motion detected in any of the X, Y, and Z-directions. In the event of an ambient vibration event, the position of the optical head may be adjusted only until the displacement of the optical head is reduced below a reduction threshold, which may be user-selected. In the event of a momentary external vibration event, the position of the optical head may be adjusted only until the displacement of the optical head is reduced below an active reduction cutoff, which may be user-selected. The steps of determining the distance and direction the optical head must be adjusted to reduce the vibration of the vibration event detected, generating, and transmitting the control signal may be performed in real time.

Method 400 may be implemented using the active vibration reduction system of FIG. 1, or any other suitable system. The preferred initialization point for such methods and the order of their steps may depend on the implementation chosen. In some embodiments, some steps may be optionally omitted, repeated, or combined. In some embodiments, some steps of such methods may be executed in parallel with other steps. In certain embodiments, the methods may be implemented partially or fully in software embodied in computer-readable media.

For the purposes of this disclosure, computer-readable media may include any instrumentality or aggregation of instrumentalities that may retain data and/or instructions for a period of time. Computer-readable media may include, without limitation, storage media such as a direct access storage device (e.g., a hard disk drive or floppy disk), a sequential access storage device (e.g., a tape disk drive), compact disk, CD-ROM, DVD, random access memory (RAM), read-only memory (ROM), electrically erasable programmable read-only memory (EEPROM), and/or flash memory; as well as communications media such wires, optical fibers, and other electromagnetic and/or optical carriers; and/or any combination of the foregoing.

The above disclosed subject matter is to be considered illustrative, and not restrictive, and the appended claims are intended to cover all such modifications, enhancements, and other embodiments which fall within the true spirit and scope of the present disclosure. For example, it may be applied to non-vibrational unintentional movements.

1. A system for active vibration reduction, comprising:
a surgical microscope with an optical head;
a control device operable to adjust a position of the optical head;
an accelerometer connected to the optical head, the accelerometer operable to detect motion of the optical head and generate data relating to the motion; and
a processor configured to determine whether the motion is an intentional movement, and ambient vibration event, or a momentary external vibration:
wherein the processor determines that the motion is an intentional movement when a displacement caused by the motion is greater than an ambient vibration boundary and when, after a first motion reversal, the displacement is greater than a normal vibration boundary,
wherein the processor determines that the motion is an ambient vibration event when the displacement caused by the motion is within ambient vibration boundary, and
wherein the processor determines that the motion is a momentary external vibration then the displacement caused by the motion is greater than an ambient vibration boundary and when, after a first motion reversal, the displacement is within the normal vibration boundary;
wherein the processor is further configured to generate a control signal and transmit the control signal to the control device, the control signal operable to cause the control device to:
adjust a position of the optical head in the distance and direction determined to reduce a vibration of the ambient vibration event until the displacement of the optical head is reduced below a reduction threshold that is less than the ambient vibration boundary, and
adjust the position of the optical head in the distance and direction determined to reduce a vibration of the momentary external vibration until the displacement of the optical head is reduced below an active reduction cutoff that is less than the normal vibration boundary.

2. The system of claim 1, wherein the accelerometer is a 3-axis accelerometer that detects and generates data relating to the motion of the optical head in the X, Y, and Z-directions.

3. The system of claim 1, wherein the accelerometer is an accelerometer that detects and generates data relating to the motion of the optical head in at least one of the X, Y, and Z-directions.

4. The system of claim 3, wherein the control signal is further operable to adjust the position of the optical head in any of the X, Y, and Z-directions.

5. The system of claim 4, wherein the position of the optical head in any of the X, Y, and Z-directions is adjusted in the direction opposite the direction of the motion detected in any of the X, Y, and Z-directions.

6. The system of claim 1, wherein determining the distance and direction the optical head must be adjusted to reduce the vibration detected, generating, and transmitting the control signal is performed in real time.
